(19) United States
(12) Patent Application Publication
(10) Pub. No.: US 2016/0213373 A1
(43) Pub. Date: Jul. 28, 2016

(54) Radial Artery Closure Device

(71) Applicants: William Joseph Drasler, Minnetonka, MN (US); Mark Lynn Jenson, Greenfield, MN (US); Richard Charles Kravik, Champlin, MN (US); William Joseph Drasler, II, Minnetonka, MN (US)

(72) Inventors: William Joseph Drasler, Minnetonka, MN (US); Mark Lynn Jenson, Greenfield, MN (US); Richard Charles Kravik, Champlin, MN (US); William Joseph Drasler, II, Minnetonka, MN (US)

(21) Appl. No.: 14/607,027

(22) Filed: Jan. 27, 2015

(51) Int. Cl. A61B 17/12

(52) U.S. Cl. CPC A61B 17/1204; A61B 17/12099

(57) ABSTRACT

A radial closure device has a compression element to place compression force against the radial access site. The invention further comprises an occlusion element that places an occlusion force against the ulnar artery to reduce blood pressure at the arteriotomy site and increase blood flow in the radial artery thereby reducing radial artery occlusion and reducing the time and compression force to achieve hemostasis. A radial restriction element can also be placed upstream of the access site to further reduce radial blood pressure at the arteriotomy site.

### CROSS REFERENCE TO RELATED APPLICATIONS

This patent application makes reference to and thereby incorporates all information found in the provisional patent application No. 61/966,485 entitled Radial Artery Closure Device, filed 24 Feb. 2014 by William J. Drasler, Mark L. Jenson, Richard C. Kravik, and William J. Drasler II.

### BACKGROUND OF THE INVENTION

The radial artery provides an alternate site for access to the vasculature for performing interventional therapeutic procedures including coronary angioplasty. It offers advantages over standard femoral access by allowing vascular closure in a vessel that is more easily closed and often allows the patient to return home on the same day of the procedure thus saving the cost of an overnight hospital stay.

The radial artery is smaller in diameter than the femoral artery and hence the introducer sheath is similar in profile to the lumen of the artery. Closure of the radial artery access site can sometimes lead to occlusion of the radial artery. Loss of the radial artery can lead to improper perfusion of the hand if the ulnar artery is not fully functional. Also, loss of radial artery patency can prohibit a repeat procedure to the patient using that radial artery.

Current radial artery closure devices apply a force or compression onto the radial artery access site. The compression can be performed via one or more inflated balloons or via a mechanical compression device applied over the radial artery access site. The compression device is often adjusted during the 1-3 hours following application of the compression device to reduce the amount of compression being applied while monitoring for maintenance of hemostasis at the access site. The compression applied initially to the access site can be somewhat painful and cause discomfort; radial artery total occlusion can occur if care is not taken to feel for a pulse in the radial artery downstream of the access site. If too much compression is applied, the radial artery can become occluded, if not enough pressure is applied, the radial artery can continue to bleed. As the compression device is adjusted, bleeding can reoccur at the access site due to movement of the compression device and this movement is also transmitted to the radial artery puncture site at the wall of the artery.

A device is needed which can provide reliable hemostasis of the radial artery without causing occlusion of the radial artery. The device should not cause discomfort to the patient. The device should not require large movement of the radial alter access site such that upon making adjustments to the device, such as removing compression force, the access site is less likely to reinitiate bleeding.

### SUMMARY

The present invention is a radial artery occlusion device that is used to close a radial artery access site used for percutaneous access to the radial artery of the arm. Percutaneous access to the radial artery is often obtained to perform therapeutic or diagnostic procedures within the body including coronary angioplasty and coronary stent placement. The invention can also be applied to other vessels of the body where it is important to ensure that vessel patency is maintained while hemostasis is being performed. Additionally, the invention has application where a second artery besides the one being accessed and closed is providing collateral blood flow to a region of tissue downstream of the arterial access site.

The arterial vasculature of the arm provides both a radial artery and an ulnar artery to deliver blood to the hand. In the region of the wrist and hand, collateral arteries join the radial artery to the ulnar artery to ensure that blood is provided to the hand from both the radial artery as well as the ulnar artery. If the ulnar artery is occluded, either totally or partially, the flow through the radial artery will increase to provide necessary blood flow to the hand; this increase in blood flow helps to prevent total occlusion or blockage of the radial artery as compression is applied to an access site in the radial artery. Occlusion of the ulnar artery, either totally or partially, also will reduce the pressure in the radial artery in a location downstream from the access site thereby requiring less compressive force at the access site to initiate hemostasis. This increased blood flow through the radial artery combined with a reduction in pressure downstream of the access site both improve the ability of an operator to properly provide hemostasis to the radial artery without causing a loss of radial artery patency. For clarity within the present patent application, compression of the radial artery shall mean the application of a force onto the surface of the forearm which thereby applies a force onto the radial artery access site toward the radial bone that stops bleeding from the arteriotomy site but does not cause occlusion or stoppage of blood flow within the radial artery. Occlusion of the ulnar artery shall mean either complete or partial occlusion and can range from complete stoppage of blood flow through the ulnar artery to a reduction in ulnar blood flow. It is understood that partial occlusion of the ulnar artery which allows at least some reduced blood flow through the ulnar artery that is less than normal ulnar blood flow can provide the benefits to hemostasis of the radial access site; such partial occlusion of the ulnar artery is also included in the present invention.

If the radial artery is restricted upstream of the access site such that the radial blood flow is reduced from normal, the pressure upstream (and in some cases downstream if no collateral circulation is present) of the access site will be less than normal radial artery pressure. This reduction in radial artery pressure at the access site allows the operator to apply less compressive force at the access site to form hemostasis. Application of less compressive force results in less movement of the access site and less movement of the arterial puncture site or arteriotomy site. As the compressive force is reduced or adjusted following application of the closure device, the access site is less likely to bleed due to a lowering of the amount of movement that occurs at the vessel access site.

One embodiment of the present invention is a radial artery closure device having a support plate positioned on the palmar side of the forearm over the radial artery access site. A radial compression surface is positioned adjacent to the radial artery access site and is located between the support plate and the forearm. A compression means serves to push the compression surface with a force against the radial access site; the compression means can comprise one or more balloons, for example that are inflated via a fluid such as air or saline; alternately the compression means can comprise a threaded screw mechanism or other mechanical mechanism. The support plate can have a curved portion that is located adjacent the palmer surface and the lateral surface of the forearm. The curved portion allows the force being applied to the radial artery access site to be delivered perpendicular to the surface of the curved portion and hence is directed toward the radius bone to provide improved back support to the radial artery for generating reliable hemostasis. The support plate can be designed such that it is adjustable in width providing a width extending from the lateral aspect (thumb side) to the medial aspect of the forearm to fit the forearm width of the patient.

Located on the support plate and facing distally (toward the anterior surface of the wrist) is an energy transducer directed toward the palmar surface of the forearm at an angle (140) of 35-45 degrees off of the surface of the forearm. One anticipated embodiment for the energy transducer is an ultrasound (US) transducer operating at a frequency of approximately 5-20 MHz. A single ultrasound transducer can emit a sound wave and receive the reflected sound wave back from the flowing blood in the radial artery. The reflected wave is altered in its frequency due to a Doppler shift which indicates that the blood is moving through the radial artery and the radial artery is therefore patent; also the Doppler shift (a drop in frequency from the emitted ultrasound wave) indicates that the blood flow is indeed moving away from the transducer and is moving distally; therefore the blood flow being observed is indeed radial artery blood flow and not venous flow. Alternately, the ultrasound transducer can have a two (or more) crystals, one for emitting the ultrasound energy and the other for receiving the ultrasound energy.

Other types of energy transducers are anticipated in the present invention to assess that blood is flowing within the radial artery and it is thereby patent. For example, one can deliver an electromagnetic energy signal that is absorbed in blood and is indicative of oxygenated blood being carried by a patent blood vessel. Such methods are often used in pulse oximeters used to identify the oxygen saturation levels in patients. In an alternate embodiment of an energy transducer heat can be detected in the radial artery downstream of the access site using IR energy emitted from the artery and absorbed on an IR receiver located on the support plate. In yet another embodiment of an energy transducer movement of the radial artery downstream of the access site can be observed via video and using digital subtraction to determine a pulse movement in the radial artery. In a further embodiment of an energy transducer auscultation can be alternately used to hear a bruits caused by turbulence of the blood flowing through an artery downstream of a restriction or narrowing in the artery at the access site. The bruits signal can be amplified and delivered to an operator as an audible or visual signal that indicates that the radial artery is patent.

An alternate embodiment for the present invention includes (in addition to the radial compression surface) an ulnar occlusion surface located adjacent the ulnar artery and held between a support plate and the anterior surface of the forearm. The support plate located adjacent the ulnar artery is parallel to the anterior surface of the forearm and extending to the medial aspect of the forearm to direct the force downward from the occlusion surface toward the ulna bone located directly below the ulnar artery in an anterior to posterior direction. An ulnar occlusion means that applies a force to the ulnar occlusion surface can comprise similar structures that are described for the radial compression means. The support plate for the ulnar compression means and ulnar support plate can be the same support plate as described for the radial support plate; alternately, a second ulnar support plate can be used to provide occlusive force to a compression or occlusion means that applies a force to the ulnar artery.

The embodiment having the radial artery compression means and the ulnar occlusion means offers the benefit of increased blood flow through the radial artery due to a lower blood pressure downstream of the access site; this increased blood flow will enhance patency of the radial artery. Also, the lower blood pressure provided downstream of the access site helps to reduce the amount of bleeding at the access site as well as reduces the amount of movement needed by the compression surface to gain hemostasis. Thus the reduction of force of the compression surface at the radial access site and subsequent removal of the compression surface from the radial artery access site after hemostasis has been established can be performed with reduced likelihood of bleeding at the access site.

Another embodiment for the present invention includes (in addition to at least the radial compression surface) a radial restriction surface located upstream of the radial compression surface and located adjacent the radial artery on an anterior surface of the forearm upstream from the access site. For the purposes of the present invention arterial restriction shall mean that blood flow through the artery is reduced but is not completely blocked or totally occluded. The radial restriction surface is held between a support plate and anterior surface of the forearm adjacent the radial artery. The support plate located adjacent the anterior surface that is adjacent the radial artery (upstream from the access site) has a curved portion around the lateral aspect of the anterior surface of the forearm to direct the force perpendicular to the curved surface from the restriction surface toward the radius bone located more medial than the radial artery. A radial restriction means that supplies a force to the radial restriction surface to hold it against the radial artery to restrict radial blood flow can comprise similar structures that are described for the radial compression means. The support plate for the radial restriction means and radial compression means can be the same support plate as described for the radial support plate; alternately, a second proximal radial support plate can be used to provide restrictive force to the radial artery upstream from the radial access site.

The embodiment having both the radial artery compression means and the radial artery restriction means provides the benefit of a reduced radial pressure upstream of the access site and hence less force being required by the compression surface to gain hemostasis. Thus, the movement of the arteriotomy site will be less and removal of the compression surface following hemostasis is less likely to cause rebleeding at the access site. The blood flow rate through the radial artery would be reduced with this embodiment; the use of an energy transducer to detect radial artery blood flow and radial artery patency enables the advantages of low radial blood flow without the potential concern that the radial artery has inadvertently become totally occluded due to the low blood flow without notifying the operator and adjustment to be made to reestablish radial artery blood flow.

In yet another embodiment, the radial artery closure device of the present invention can include the radial compression means, the ultrasound energy transducer, the ulnar artery occlusion means, and the radial restriction means located upstream of the access site. With this system, the advantages of a high radial blood flow and low radial blood pressure downstream of the access site (due to ulnar occlusion, either totally or partially) combined with a low upstream pressure (due to restricting the radial artery upstream) within the radial artery provide an improved radial artery patency, a low likelihood for access site bleeding, and an ease of removal of the compression surface without rebleeding. The ultrasound transducer serves to ensure that radial blood flow is maintained during the procedure and notifying the operator if radial blood flow has been inadvertently blocked or stopped.

Methods of use are also described wherein hemostasis of the radial artery is obtained while ensuring that radial artery patency is maintained. The methods include the use of a radial artery compression means along with an ultrasound transducer. The methods can further include the use of an ulnar occlusion means or a radial artery restriction means located upstream from the access site. The methods can include the radial artery compression means, the ulnar occlusion means, the radial restriction means, and the ultrasound transducer.

It is understood that even though the description is directed toward an ultrasound energy transducer, any type of energy transducer that is able to detect flow in the radial artery and also ulnar artery if desired can be used with the present invention.

### BRIEF DESCRIPTION OF THE DRAWINGS

FIG. 1 is an anatomical depiction of the vasculature of the forearm and hand with the radial artery, ulnar artery, collateral arteries, capillaries, and venous system and showing a compression surface adjacent the access site.

FIG. 2 is an anatomical depiction of the vasculature of the forearm and hand showing a compression surface adjacent the radial artery access site and an occlusion surface over the ulnar artery.

FIG. 3 is an anatomical depiction of the vasculature of the forearm and hand showing a compression surface adjacent the radial artery access site and a restriction surface over the radial artery upstream of the access site.

FIG. 4 is an anatomical depiction of the vasculature of the forearm and hand showing a compression surface adjacent the radial artery access site, an occlusion surface over the ulnar artery, and a restriction surface over the radial artery upstream of the access site.

FIG. 5 is a plan view of an anterior surface of a forearm having a support plate with a compression means and an energy transducer located on the anterior surface.

FIG. 6 is a plan view of the medial aspect of a forearm having a compression means located above the radial artery access site.

FIG. 7 is a cross-sectional view through the forearm showing the support plate of the closure device on the anterior aspect of the forearm.

FIG. 8 is a cross-sectional view through the forearm showing the energy transducer directing an energy signal onto the radial artery.

FIG. 9 is a partial longitudinal view through a forearm through the radial artery showing the energy signal directed from the energy transducer at an angle toward the radial artery downstream of the arteriotomy.

FIG. 10 is a plan view of the anterior aspect of a forearm showing a support plate having a compression means located above the radial artery and an occlusion means located above the ulnar artery.

FIG. 11 is a cross-sectional view through a forearm showing the compression means placing a force onto the radial artery and an occlusion means placing a force onto the ulnar artery.

FIG. 12 is an anterior view of a forearm showing one support plate with the compression means over the radial artery access site and a second restriction support plate with the restriction means over the radial artery upstream of the radial artery access site.

FIG. 13A is a medial view of a forearm showing a compression means and a restriction means located above the radial artery; the compression means located above the radial access site and the restriction means located upstream of the radial access site.

FIG. 13B shows a cross-section of a forearm having a radial restriction plate located over the radial artery but upstream of the radial access site.

FIG. 14 is an anterior view of a forearm having a distal support plate that contains the radial compression means, the ulnar occlusion means, and the energy transducer; a second support plate located proximally contains the radial restriction means.

FIG. 15 is an anterior view of the forearm have a single support plate that contains a compression means, an occlusion means; the compression means and occlusion means are located distally from the restriction means.

FIG. 16 is an anterior view of the forearm having a single support plate that contains a compression means, an occlusion means; the compression means is located distally from the occlusion means and the restriction means.

FIG. 17 is an anterior view of a forearm having a distal support plate that contains the radial compression means and the energy transducer; a second support plate located proximally contains the radial restriction means the ulnar occlusion means.

FIG. 18 is a cross-sectional view of the forearm showing a balloon compression means located above the radial artery.

FIG. 19 is a cross-sectional view of the forearm showing an inner compression balloon adjacent the access site and an outer balloon that extends across the anterior aspect of the forearm adjacent the support plate.

FIG. 20 shows one compression balloon in contact with the radial artery adjacent the access site and a second occlusion balloon located on top of the ulnar artery.

FIG. 21 shows a small compression balloon in contact with the radial artery adjacent the access site and a second small occlusion balloon located on top of the ulnar artery; a larger outer balloon is located anterior to the small balloons and adjacent the support plate.

### DETAILED DESCRIPTION

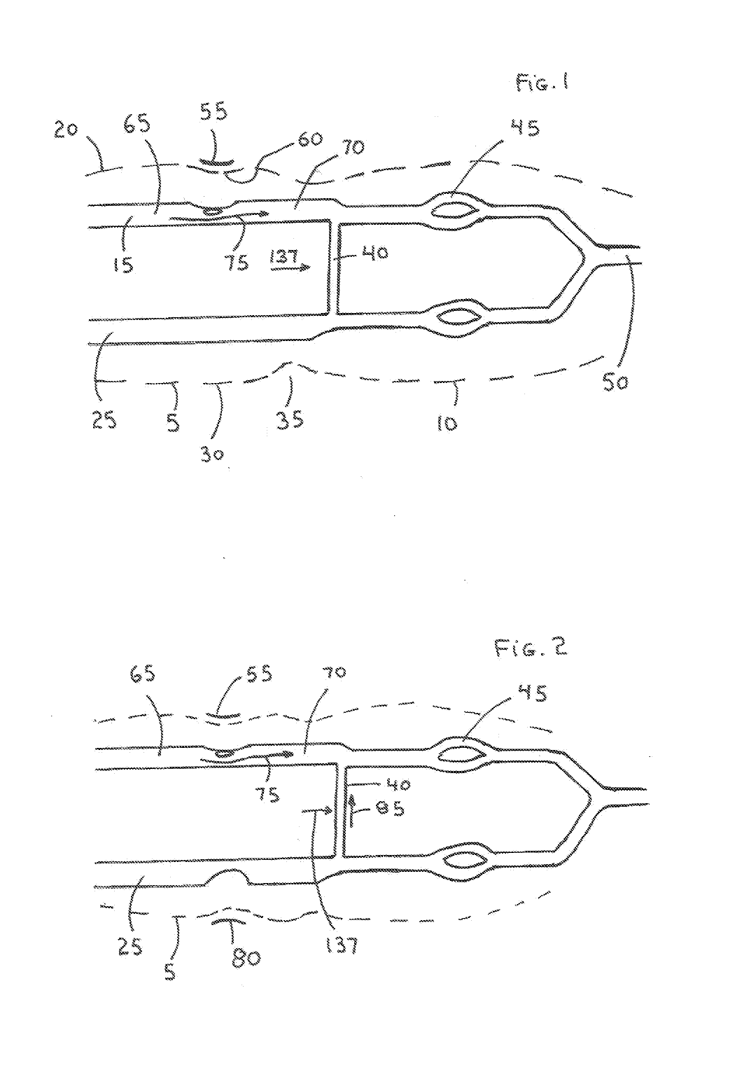

The vasculature of the forearm (5) and hand (10) can be modeled for our use as shown in FIG. 1. The radial artery (15) runs along the forearm (5) on the lateral side (20) (thumb side) and the ulnar artery (25) runs along the medial side (30) of forearm (5) delivering blood to forearm (5) tissues and the hand (10). At the wrist level (35) and in hand (10) region there are collateral arteries (40) that connect the radial artery (15) with the ulnar artery (25); these collateral arteries (40) include the palmar carpal branch, the dorsal carpal arch, the superficial palmar arch, and the deep palmar arch. These collateral arteries (40) are modeled in FIG. 1 as a single collateral artery (40). If the radial artery (15) were to become totally occluded, blood would be supplied to the hand (10) via the ulnar artery (25). Arterial blood reaching the hand (10) is then directed through capillaries (45) and into the venous system (50). As shown in FIG. 1 a compression surface (55) has been placed over an access site (60) above the radial artery (15) resulting in an upstream pressure (65), Pu, just upstream of the access site (60) and a downstream pressure (70), Pd, downstream (137) of the access site (60). The radial blood flow (75) in the radial artery (15) is controlled by the pressure difference (Pu−Pd) between the upstream pressure (65) upstream, Pu, and the downstream pressure (70), Pd.

Upon application of an occlusion, either total or partial, to the ulnar artery (25) (along with the compression surface (55) located at the access site (60)) via an occlusion surface (80) as shown in FIG. 2 the downstream pressure (70) in the radial artery will be reduced from its normal pressure without the ulnar occlusion surface (80) due to blockage of collateral blood flow (85) through the collateral arteries (40). The ulnar occlusion results in an increase in radial blood flow (75) through the radial artery (15) in comparison to radial blood flow (75) found without the ulnar occlusion. This increase in radial blood flow (75) will enhance the ability of the radial artery (15) to remain patent; it will also reduce the average pressure (i.e., (Pu+Pd)/2) at the access site (60) thereby reducing the propensity for access site bleeding. It is understood that partial occlusion of the ulnar artery (25) can be provided to gain some or all of the benefits provided by total ulnar occlusion. An ultrasound energy device (135), detector, or transducer can be directed at the ulnar artery (25) downstream (137) of the detector, for example, to ensure that ulnar blood flow is either totally or partially occluded.

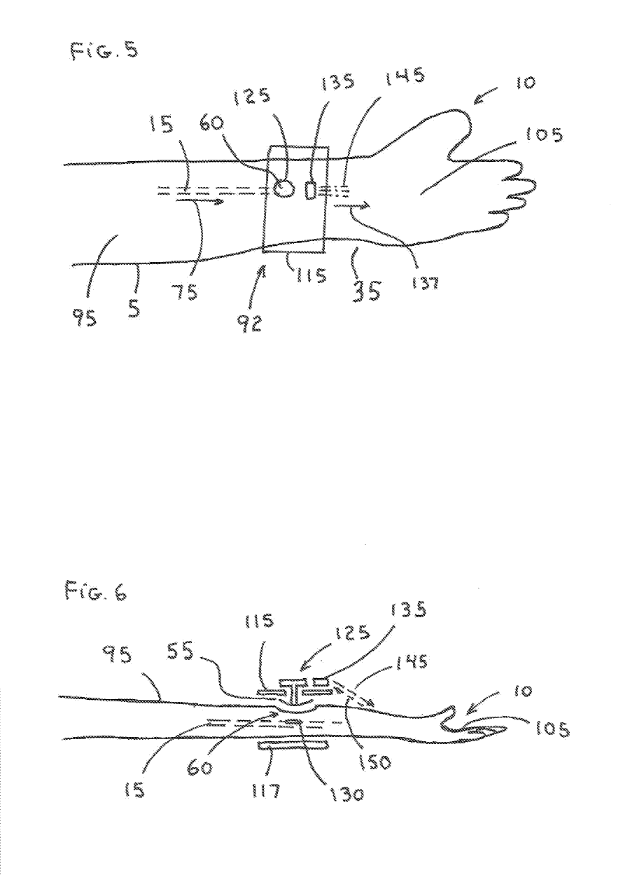

Upon application of a restriction via a restriction surface (90) to the radial artery (15) upstream (136) from the access site (60) as shown in FIG. 3 (along with the compression surface (55) located at the access site (60)), the upstream pressure (65), Pu, located upstream of the access site (60) is reduced from normal radial blood pressure without the upstream restriction surface (90). The downstream pressure (70), Pd, can be maintained at a normal downstream (137) radial artery (15) blood pressure due to the collateral blood flow (85), although the downstream pressure (70), Pd, can drop somewhat from normal if the collateral blood flow (85) is not high enough to maintain normal radial downstream pressure (70). This result of the restriction surface (90) is a lowering of radial blood flow (75). The reduced average pressure, (Pu+Pd)/2, at the access site (60) provides a reduced propensity for bleeding at the access site (60). The reduced driving pressure (Pu−Pd) across the access site (60) leads to a lower radial blood flow (75). The reduction of radial blood flow (75) can be monitored using an energy transducer (135) (see FIG. 5) as described in the embodiments of the invention to ensure that radial blood flow (75) is maintained and notification to the operator via audible or visual alarm is provided by the energy transducer (135).

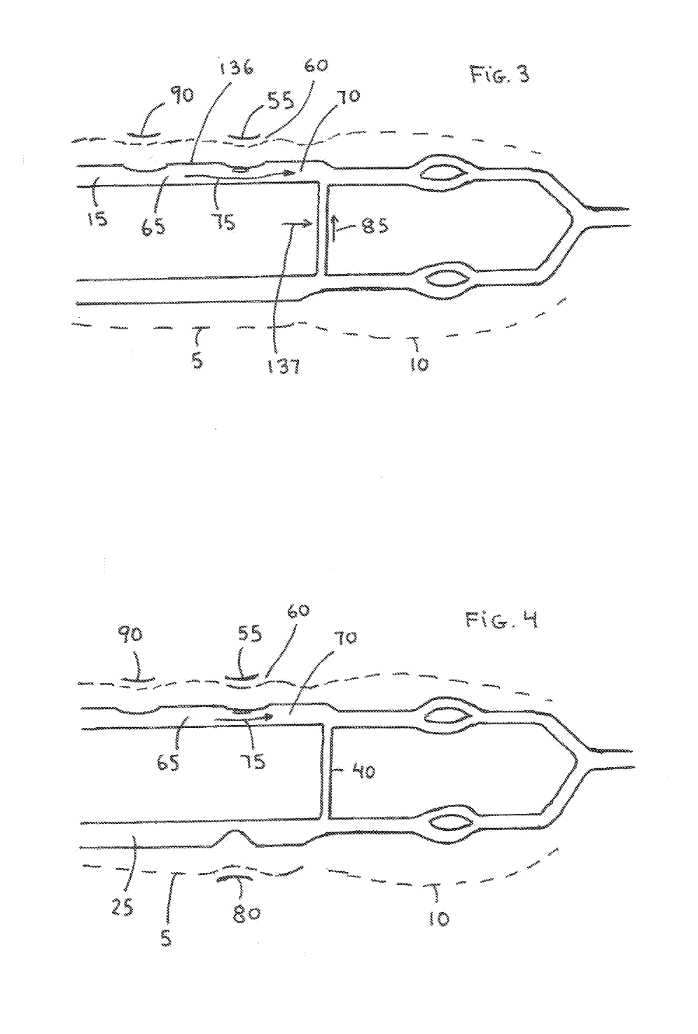

As shown in FIG. 4 application of a restriction surface (90) upstream from the access site (60) along with an occlusion surface (80) on the ulnar artery (25) (along with the compression surface (55) located at the access site (60)) provides a low downstream pressure (70), Pd, due to occlusion (either total or partial) of the ulnar artery (25), and a low upstream pressure (65), Pu, due to restriction of the radial artery (15) upstream of the access site (60). The result is that the average pressure at the access site (60) (Pu+Pd)/2 is very low and hence bleeding is not likely and hemostasis is easy to establish with minimal or reduced compression at the access site (60). The radial blood flow (75) rate is determined by the driving force (Pu−Pd); assurance that the radial artery (15) remains patent can be accomplished by use of an energy transducer (135) as described in the embodiments of the invention via a signal that notifies the operator of an occluded radial artery (15).

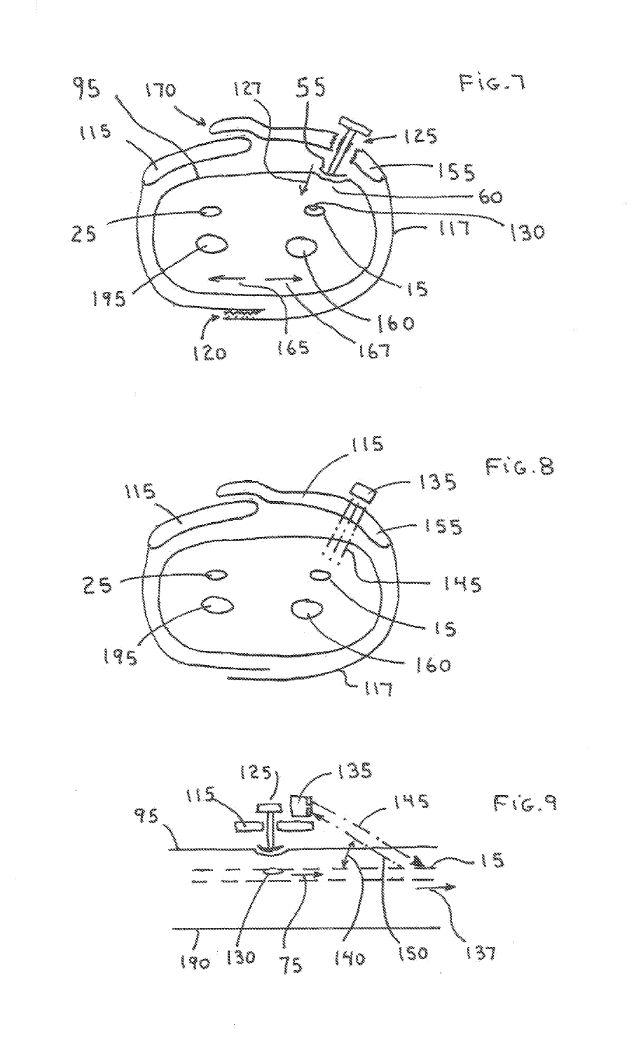

One embodiment of the closure device (92) of the present invention is shown in FIGS. 5-9. FIG. 5 show the anterior surface (95) of the left forearm (5) and the palmar surface (105) of the hand (10) having a support plate (115) extending across all or part of the anterior surface (95) of the forearm adjacent and above the access site (60), but at least extending across the access site (60) located in the radial artery (15) near (1-6 inches away from) the wrist (135) of the patient. FIG. 6 shows a medial view of the left forearm (5) with the support plate (115) located on the anterior surface (95) with palmar surface (105) of the hand facing upwards. A compression surface (55) is located between the support plate (115) and the anterior surface (95) of the forearm (5). A holding strap (117) with Velcro or other attachment means (120) (see FIG. 7) holds the support plate (115) against the anterior surface (95) of the forearm (5). A compression means or compression element (125) applies a force (127) onto the compression surface (55) to generate a compression force (127) against the access site (60) to provide hemostasis of the radial artery (15) arteriotomy (130) at the access site (60). The compression means or element (125) can be a threaded screw, one or more balloons, or other means to apply force onto the compression surface (55) to force it into contact with the anterior surface (95) of the forearm (5); the compression element is adjustable to provide an adjustable compression force onto the ulnar artery. A forward or distally directed energy transducer, energy detector, or energy device (135) is directed toward the radial artery (15) distal to or downstream (137) of the access site (60) and arteriotomy (130). A cross section through the forearm (5) at the location of the energy transducer (135) is shown in FIG. 8; the ultrasound energy (or other energy form) is directed from the energy transducer (135) located on the support plate (115) toward the radial artery (15). The energy transducer (135) directs an energy form at an angle (140) of approximately a 30-45 degrees (range 20-60 degrees) off of the anterior surface (95) of the forearm (5) toward the radial artery (15) as shown in FIG. 9. The energy device (135) can be an ultrasound transducer (ultrasound), light, electromagnetic, magnetic, thermal, visual, auscultation, or other means to detect radial blood flow (75) in the radial artery (15).

For an ultrasound transducer, for example, an ultrasound delivery signal (145) of frequency 5-20 MHz can be directed from a piezoelectric crystal toward the radial artery (15) downstream (137) from the arteriotomy (130). Due to radial blood flow, a reflected signal (150), as shown in FIG. 9, is received by the energy transducer (135) with a shift in frequency due to flow velocity in the radial artery (15) moving downstream (137) of the access site (60). The receipt of a reflected signal (150) with a Doppler shift in frequency (lower frequency for the reflected signal (150) indicates that blood is flowing and it is flowing away or downstream (137) from the energy transducer (135). The presence of the ultrasound energy transducer (135) or other energy transducer (135) allows the patency of the radial artery (15) to be evaluated such that the radial artery (15) does not occlude during the closure procedure; adjustment can be made to the compression surface (55), the occlusion surface (80), or the restriction surface (90) to reestablish radial blood flow (75). For example, the radial restriction surface (90) can be loosened, adjusted, or removed to enhance radial blood flow (75); reduced compression via loosening the compression surface (55) at the access site (60) may also improve radial blood flow (75). If the ulnar artery has become totally occluded for too long a period of time (i.e., from several minutes to over an hour) the ulnar occlusion surface can be loosened, adjusted, or removed. If the energy transducer (135) detects, for example, that radial blood flow (75) has ceased, a light or audible signal can be given to the operator and adjustments to the closure device (92) can be made, such as adjusting the force applied by the compression surface (55), the occlusion surface (80), or the restriction surface (90). It is understood that an ultrasound transducer (135) can also be placed above the ulnar artery (25) and directed toward the ulnar artery (25) distal to the ultrasound transducer (135) to detect if the ulnar artery is totally occluded, partially occluded, or fully patent, as desired.

The support plate (115) can have a curved portion (155) as shown in FIG. 7 such that the compression means (125) directs a compression force (127) perpendicular to the curved portion (155) thereby forcing the radial access site (60) above the radial artery (15) into hemostasis as the radial artery (15) is supported on its posterior side by the radius bone (160); the radius bone (160) is located in a more medial direction (165) than the radial artery (15). Directing the compression surface (55) with a component of the compression force (127) in a medial direction (165) can alternately be accomplished by orienting the compression means or occlusion element (125) to direct the compression surface (55) in a medial direction (165), even if, for example, the compression surface (55) does not have a curved portion (155) and is, for example, a generally planar configuration.

The support plate (115) as shown in FIG. 7 has an adjustment means (170); the adjustment means (170) allows the width of the support plate (115) from medial to lateral direction (167) to be varied to match the width of the patient forearm width (175).

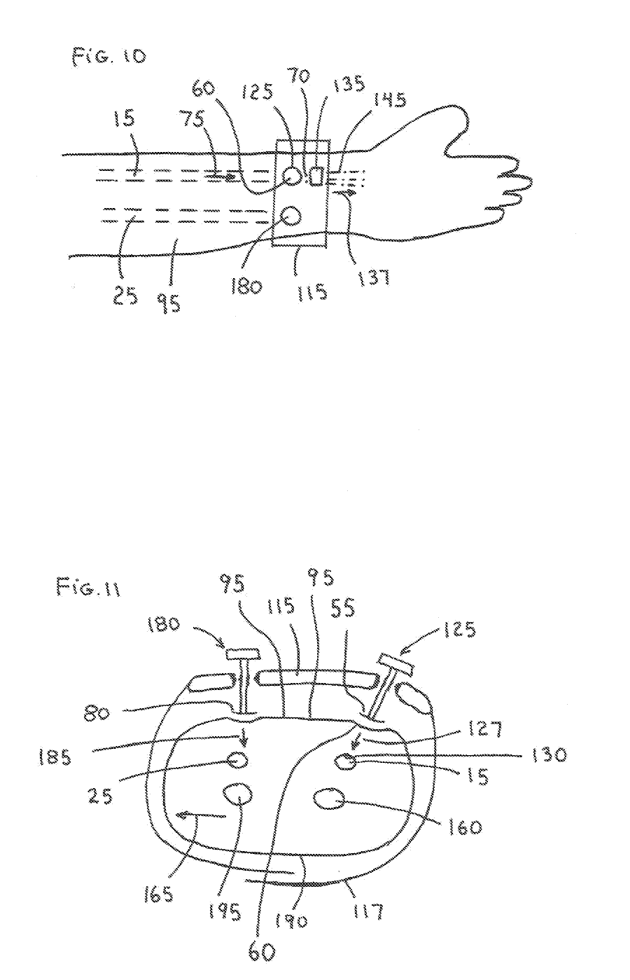

Another embodiment of the present invention is shown in FIGS. 10 and 11. In addition to the elements found in FIGS. 5-9, this embodiment also has an ulnar occlusion means or occlusion element (180) attached to the support plate (115). The ulnar occlusion means or element (180) applies an occlusion force (185) to the ulnar artery (25) via an ulnar occlusion surface (80) that is located between the support plate (115) and the anterior surface (95) of the forearm (5). The occlusion element can be adjusted to alter the amount of occlusion force (185) that is applied to the ulnar artery. The ulnar occlusion surface (80) is located on the anterior surface (95) adjacent the ulnar artery (25) along a region of the forearm (5) near but in a medial direction (165) with respect to the radial arteriotomy site (130). The ulnar occlusion means (180) applies an occlusion force (185) downward from the anterior surface (95) towards the posterior surface (190) to push the ulnar occlusion surface (80) against anterior surface (95) of the forearm (5) to push the ulnar artery (25) against the backstop of the ulna bone (195). The occlusion means or occlusion element (180) can be a threaded screw, one or more balloons, or other mechanical means to apply an adjustable force onto the occlusion surface (80) to force it into contact with the anterior surface (95) of the forearm (5) above the ulnar artery (25).

Occlusion, either total or partial, of the ulnar artery (25) results in a lower downstream pressure (70) in the radial artery (15) downstream (137) of the access site (60) in the direction of radial blood flow (75). This lower downstream pressure (70) allows the radial blood flow (75) to increase thereby increasing the likelihood for maintaining radial artery (15) patency. The lower downstream pressure (70) located downstream (137) of the access site (60) also enhances the ability to obtain hemostasis of the radial access site (60) due to a lower average radial artery (15) pressure, (Pu+Pd)/2, at the access site (60). The lower downstream pressure (70) also allows less movement of the access site (60) and arteriotomy site (130) thereby allowing adjustments to be made to the compression force (127) of the compression surface (55) and removal of the closure device (92) and compression surface (55) without causing rebleeding at the access site (60).

In one embodiment, as shown in FIGS. 10 and 11, the ulnar occlusion surface (80) and occlusion means or element (180) are located on the same support plate (115) as the radial compression means (125) and radial compression surface (55).

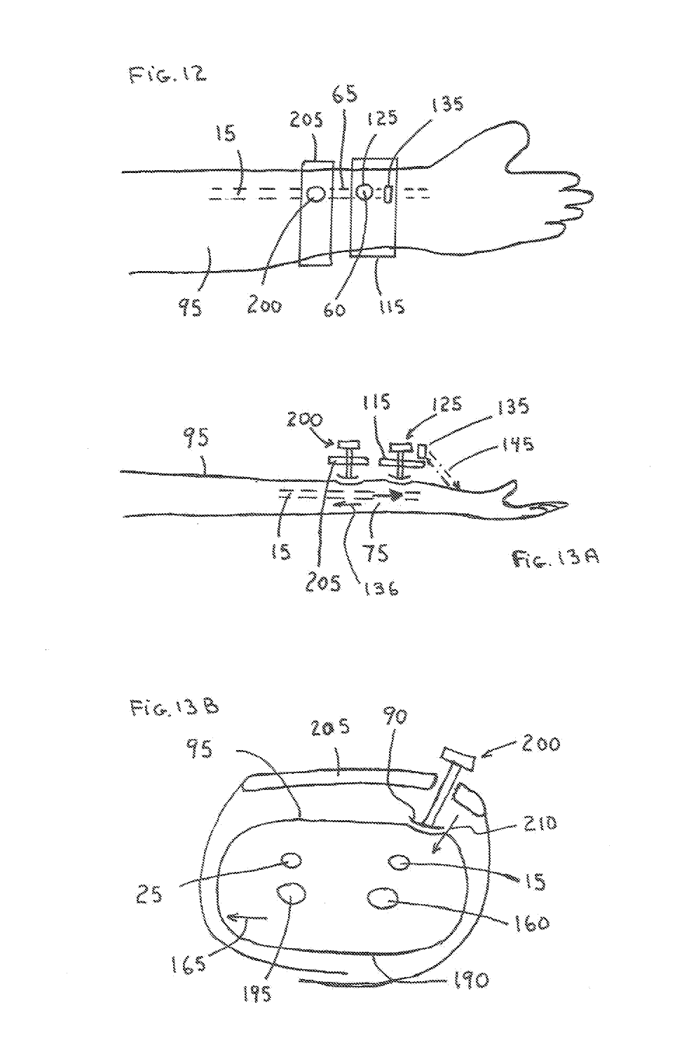

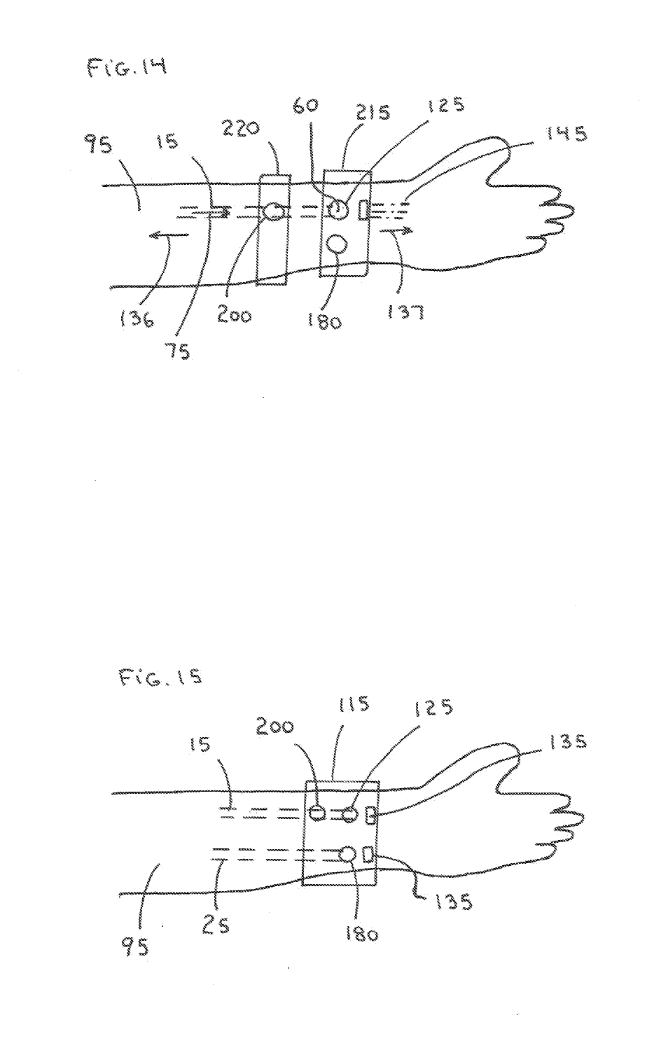

In yet another embodiment as shown in FIGS. 12-13B a radial restriction means (200) is located on a radial support plate (205) at a location upstream from the access site (60). The radial support plate (205) for the radial restriction means (200) can be a separate radial restriction support plate (205) that holds the radial restriction means (200) as shown in FIGS. 10 and 11 or a single support plate (115) can be used to support both the radial compression means (125) and the radial restriction means (200). The radial restriction support plate (205) can have a curved portion (155) to direct the restriction force (210) onto the radial restriction surface (90) with at least some component in the medial direction (165) toward the radius bone (160) as seen in FIG. 13B. The radial restriction means (200) applies a restriction force (210) via a radial restriction surface (90) located between the support plate (115) and the anterior surface (95) of the forearm (5) to the radial artery (15) upstream of the access site (60). The structural elements of the radial restriction means or radial restriction element (200) can be the same as those described for the radial compression means (125), i.e. the radial restriction means or element (200) can be a threaded screw, one or more balloons, or a mechanical compression means. The restriction means is adjustable to provide an appropriate amount of restriction force to the radial artery without causing total occlusion. The radial restriction surface (90) applies a restriction force (210) toward the posterior surface (190) of the forearm and medial direction (165) to push the radial artery (15) toward the radius bone (160) and towards the posterior surface (190) to cause a restriction in the radial artery (15) upstream (136) of the access site (60). This restriction force (210) does not occlude the radial artery (15). The radial artery (15) restriction force (210) serves to reduce radial blood flow (75) and lower radial upstream pressure (65) located upstream (136) of the access site (60). This lower radial upstream pressure (65) will allow hemostasis at the radial access site (60) to be accomplished easier. Adjustments made to the compression surface (55) at the access site (60) can be made without causing rebleeding since the movement of the access site (60) and the arteriotomy site (130) is less due to the lower radial upstream pressure (65) located upstream of the access site (60).

The radial blood flow (75) through the radial artery (15) as shown in FIGS. 12, 13A, and 13B will be lower than that without the radial restriction surface (90) being applied. The presence of an ultrasound or energy transducer (135) located on the support plate (115) ensures that radial artery (15) patency is maintained. A signal for the ultrasound or energy transducer (135) (or other energy transducer (135)) notifies the operator that radial blood flow (75) is no longer present and that action should be taken to restore radial blood flow (75) (such as reducing the restriction force (210) or compression force (127)).

FIGS. 14-17 show embodiments of the present invention having one or more support plates with a radial compression means (125) for stopping bleeding at the access site (60), an ulnar occlusion means (180) for occluding the ulnar artery (25), a restriction means (200) for restricting the radial artery (15) upstream of the access site (60), and an energy transducer (135) directed distally at the radial artery (15). The individual elements of these embodiments are as described in the previous embodiments. The advantages of this embodiment is that occlusion of the ulnar artery (25) via an occlusion surface (80) helps increase blood flow in the radial artery (15) and lower blood pressure downstream (137) of the access site (60); restriction of the radial artery (15) via a restriction surface (90) upstream of the access site (60) lowers blood pressure just upstream of the access site (60). The result is a lower radial blood pressure both upstream (136) and downstream (137) of the access site (60) to reduce bleeding and allow hemostasis with minimal compression force (127) applied to the access site (60). The radial blood flow (75) in the radial artery (15) is moderate because the radial downstream pressure (70) has been reduced due to occlusion, either total or partial, of the ulnar artery (25). The presence of an ultrasound transducer or other energy transducer (135) directed at the radial artery (15) downstream (137) of the access site (60) provides assurance that the radial artery (15) is maintained in a patent condition.

As shown in FIGS. 14-17 the support plate can take on several configurations. In FIG. 14 the radial compression means (125) and the ulnar occlusion means (180) are located on a distal support plate (215) and the radial restriction means (200) is located on a proximal support plate (220). The occlusion means (180) places a downward force onto the occlusion surface (80); the occlusion means (180) can be a threaded screw, one or more balloons, or other means to apply force onto the occlusion surface (80) (see FIG. 11) to force it into contact with the anterior surface (95) of the forearm (5) above the ulnar artery. The restriction means (200) places a downward force via the restriction surface onto the radial artery (15) upstream of the access site (60); the restriction means (200) can be a threaded screw, one or more balloons, or other means to apply a restriction force onto the restriction surface (90) to generate a restriction force (210) onto the anterior surface (95) of the forearm (5) above the radial artery at a location upstream from the access site.

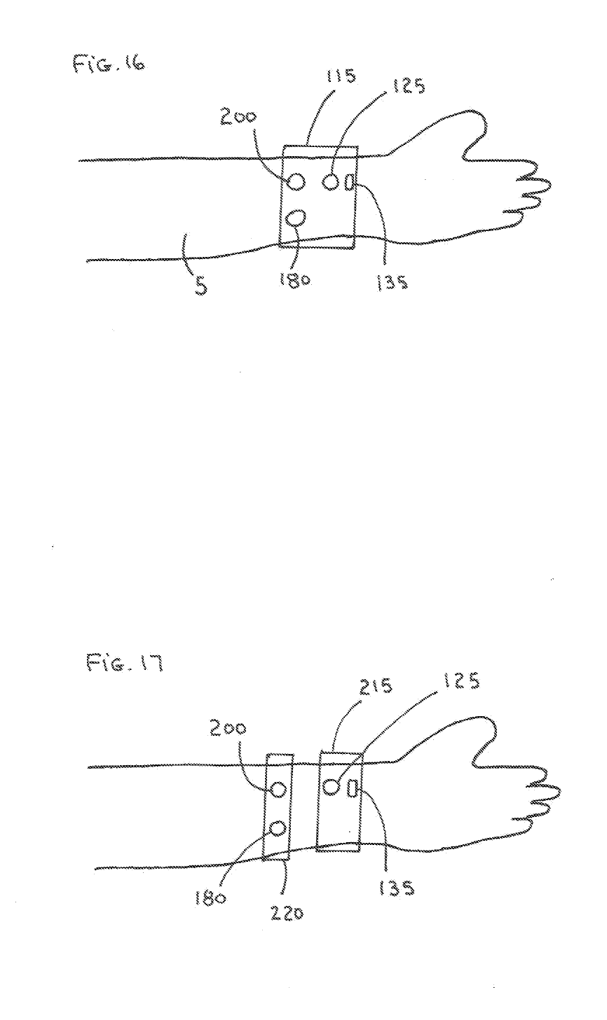

In FIGS. 15 and 16 all three means, i.e. radial compression means (125), ulnar occlusion means (180), and radial restriction means (200) are all located on a single support plate (115) along with the energy transducer (135). As shown in FIG. 15, the radial compression means (125) is located more distally on the forearm and in the same cross-sectional plane as the ulnar occlusion means (180); the radial restriction means (200) is located more proximally. As shown in FIG. 16, the radial restriction means (200) is located more proximally on the forearm (5) and in the same cross-sectional plane through the forearm (5) as the ulnar occlusion means (180); the radial compression means (125) is located more distally. As shown in FIG. 15, a second energy device (135) can be placed on the support plate (115) to direct an energy signal (145) onto the ulnar artery to assess if the ulnar artery (25) is partially occluded, totally occluded, or fully patent.

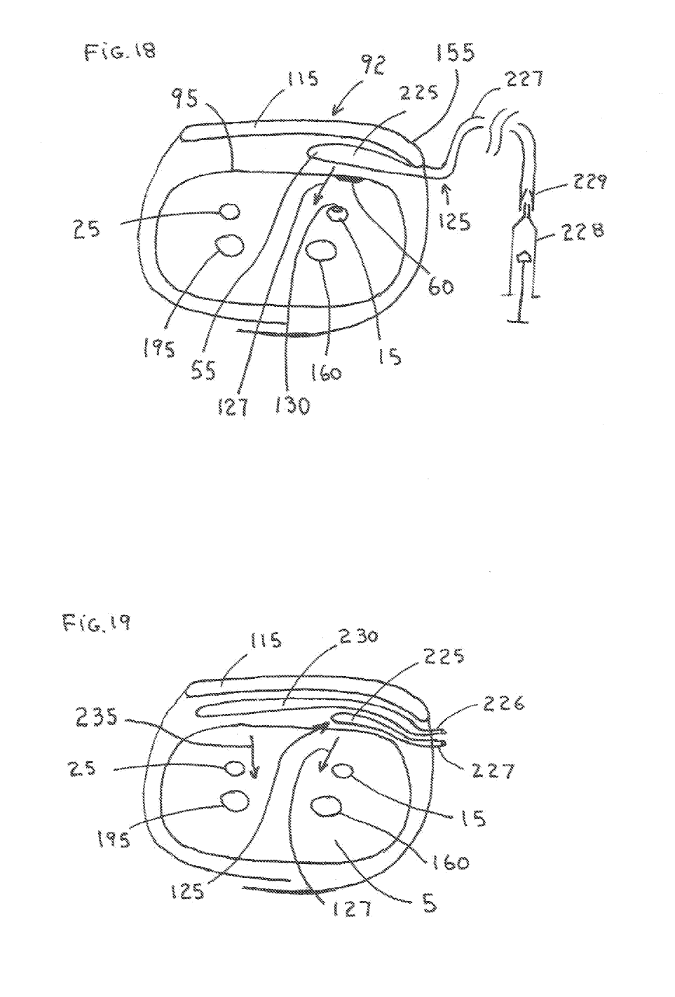

In FIG. 17 the radial compression means (125) is located on a distal support plate (215); the ulnar occlusion means (180) and radial restriction means (200) are located on a proximal support plate (220). The various arrangements allows the operator to use for example only a radial compression means (125) or a radial compression means (125) and an ulnar occlusion means (180), or any combination of the three means.

Previous embodiments have shown the radial compression means (125), the ulnar occlusion means (180), and the radial restriction means (200) as a threaded screw that applies a force via a surface such as the compression surface (55), for example to an artery of the forearm (5). It is understood that several mechanical, pneumatic, or hydraulic mechanisms can be used to apply force to the radial artery (15) or ulnar artery (25). For example, as shown in FIGS. 18-21 a balloon filled with air or saline can be used to apply a force to an artery. In FIG. 18, for example, a single compression balloon (225) is placed between the support plate (115) and the anterior surface (95) of the forearm (5) adjacent the access site (60) to provide the compression means (125) for the closure device (92). The compression balloon (225) can be filled during use via a compression balloon fill tube (227) connected to a syringe (228). A sealing valve (229) such as a duck-bill valve can be used to maintain pressure within the balloon during use. The sealing valve (229) can be released to deflate the compression balloon (225) partially to reduce the compression force (127) applied to the access site during the use of the closure device (92). The sealing valve (229) can release all pressure within the compression balloon at the completion of the access site closure procedure.

Inflation of the compression balloon (225) with air allows a compression force (127) to be applied via the lower surface of the compression balloon (225) which form a compression surface (55) against the access site (60) and against the arteriotomy site (130) of the radial artery (15). For a curved portion (155) of the support plate (115), the direction of the applied compression force (127) will be perpendicular to the curved surface and will direct the force toward the medial portion of the forearm (5) to push the radial artery (15) against a backstop of the radius bone (160) as described in earlier embodiments of the invention.

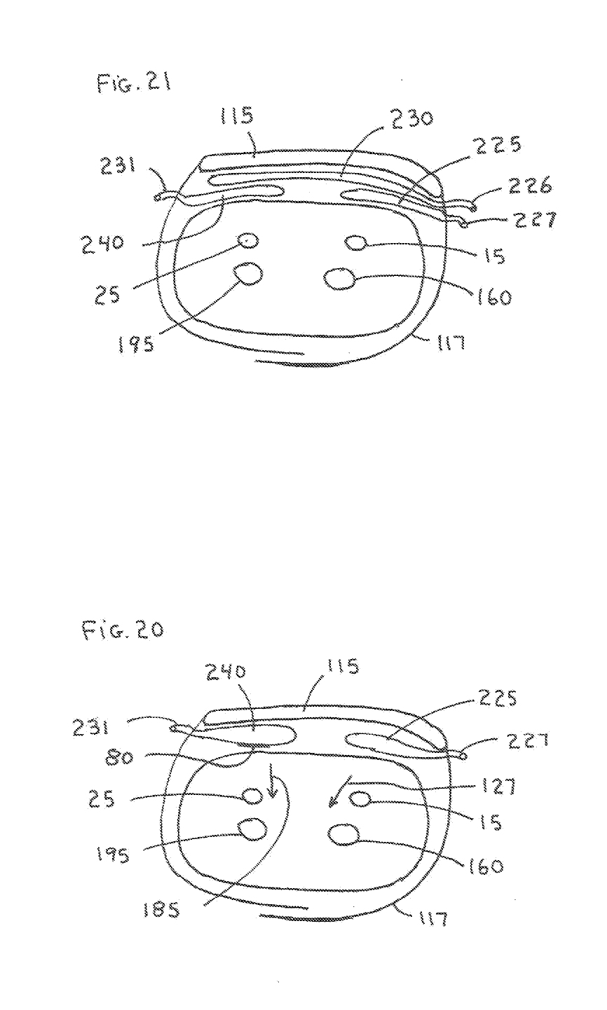

As shown in FIG. 19, two balloons can form the compression means (125) and can be used to generate a compression force (127) against the radial artery (15). It is understood that the two balloons shown in FIG. 19 could equally be used to apply a restriction force (210) to the radial artery (15) upstream of the access site (60) as described in earlier embodiments to form a restriction means or restriction element (200). The outer balloon (230) of FIG. 19 provides direct contact with the support plate (115) and applies a downward force in a posterior direction (235) to provide a pressurized backstop for the compression balloon (225). The outer balloon (230) can have an outer balloon fill tube (226) that allows it to be filled with a fluid during use and has provisions similar to the compression balloon to allow for adjustment of fluid pressure within the outer balloon (230); the outer balloon (230) extends along the support member on the anterior surface (95) of the forearm (5) above the radial artery (15) and the ulnar artery (25).

Alternately, as shown in FIG. 20 two balloons can be used, a compression balloon (225) to apply a compression force (127) to the radial artery (15) and an occlusion balloon (230) to apply an occlusion force (185) to the ulnar artery (25). The occlusion balloon (240) then becomes part of the occlusion means (180) for the closure device (92); the lower surface of the occlusion balloon (240) is the occlusion surface (80). The compression balloon (225) and occlusion balloon (240) can be inflated independently and can be adjusted independently to provide an occlusion of the ulnar artery (25) and compression of the radial artery (15). The forces applied by such balloons can be made to vary over time, for example, by creating a controlled leak or by allowing creep to occur in the balloon material. The occlusion balloon (240) can have an occlusion balloon fill tube (231) that allows for fluid entry into the occlusion balloon (240), maintenance of fluid pressure, and release of fluid pressure from within the occlusion balloon (240) similar to that shown for the compression balloon (225).

As shown in FIG. 21 another embodiment for the radial compression and ulnar occlusion means (180) is formed by three balloons; a single outer balloon (230) that extends across the entire support plate (115) and two smaller balloons, the compression balloon (225) and the occlusion balloon (240) that are directed more specifically to apply appropriate pressure to the radial or ulnar artery (25) to ensure maintenance of radial artery (15) patency, for example, while providing for ulnar artery (25) occlusion. It is understood that such balloons can be utilized in any combination to provide compression and occlusion to the radial and ulnar arteries. It is further understood that a restriction means (200) can also be constructed using the balloons described in FIGS. 18-21. The structure for the compression balloon (225) shown in FIG. 18 can be used to construct a restriction balloon, for example, that is used to restrict radial blood flow (75) upstream of the access site (60); a separate fill tube can be used to provide fluid to the restriction balloon. The outer balloon (230) structure can also be used along with a restriction balloon in a manner similar to that described for the compression balloon (225). It is understood that the compression balloon (225), the outer balloon (230), the occlusion balloon (240), or a restriction balloon used as the restriction means (200) can each have a separate compression fill tube (227), occlusion fill tube (231), outer fill tube (226) and a restriction means (200) fill tube such that each balloon is individually controlled for fluid pressure, or two or more balloons can have a common fill tube such that two or more balloons can be filled at the same time to the same pressure via a syringe (228).

The reference numerals used to describe a component used in an embodiment of the present invention can be equally used to describe components found in other embodiments of the present invention. It is understood that the present invention is not limited to embodiments presented herein and that other embodiments have also been contemplated.

The method for use for the present invention can vary depending upon whether the ulnar occlusion is used alone with the radial compression, the radial restriction is used along with radial compression, or ulnar occlusion and radial restriction are both used with radial compression. Also, radial compression used along with the energy transducer (135) is also a viable option to ensure radial patency. The method of generating hemostasis of the radial artery (15) using standard radial closure devices can take from 30 minutes to over 3 hours. The methods described in the present invention are intended to reduce hemostasis times by approximately 1 hour to a hemostasis time of 10 minutes to less than 2 hours. The benefits are due to less movement at the arteriotomy site (130) due to a lowered amount of radial artery compression force (127) required by the present invention to achieve hemostasis. The lower force requirement is due to a lowering of radial blood pressure at the arteriotomy site (130).

In one method, with the introducer sheath still in place, the ulnar artery (25) is occluded (either totally or partially); then the introducer sheath is withdrawn from the radial artery and blood flow is stopped using the radial compression means (125). After a period of time ranging from minutes to over an hour, a reduction in radial compression is performed. The ulnar artery (25) is then unoccluded while monitoring to ensure that bleeding has not occurred at the access site (60). Finally, the compression means (125) is removed to complete the hemostasis. Monitoring of the patency of the radial artery (15) is performed continuously using an ultrasound transducer directed distally onto the radial artery (15).

In an alternate method a radial artery (15) restriction is placed upstream of the radial artery (15); then the radial artery sheath is removed and bleeding is stopped at the access site (60) via the compression means (125). Over time the compression means (125) is reduced in its applied force. The restriction means (200) located upstream on the radial artery (15) is then removed. Finally, the compression means (125) is removed to complete hemostasis of the radial access site (60). Monitoring of the patency of the radial artery (15) is performed continuously using an ultrasound transducer directed distally onto the radial artery (15).

In yet an alternate method, the ulnar artery (25) is occluded with the occlusion means (180); the radial artery sheath is removed and bleeding is stopped at the access site (60) using the compression means (125). A restriction means (200) is placed upstream on the radial artery (15). Over time a reduction of compression force (127) is made at the access site (60). The ulnar occlusion is then reduced in occlusive force or released; the restriction upstream on the radial artery (15) is then loosened, reduced in restriction force, or released. Finally the compression means (125) is reduced in compression force or removed from the access site (60) to complete the hemostasis procedure. Monitoring of the patency of the radial artery (15) is performed continuously using an ultrasound transducer directed distally onto the radial artery (15). Alterations in the compression means (125), the restriction means (200), or the occlusion means (180) are performed as needed to ensure radial artery (15) patency is maintained.

1. A radial artery closure device for providing hemostasis to a radial artery access site, said closure device comprising;
A. a first support plate having a compression surface attached thereto, said compression surface being attached to a compression element that supplies a compressive force to said compression surface to compress the radial artery access site without totally occluding blood flow in the radial artery, said compressive surface providing hemostasis at the radial access site,
B. A holding strap to hold said first support plate such that said compression surface is adjacent the radial artery access site,
C. a second surface attached to a second support plate, said second surface being attached to a second element that supplies a second force to said second surface to at least partially occlude blood flow in a second artery other than the radial artery, the second artery being connected to the radial artery via collateral arteries downstream of the access site.

2. The closure device of claim 1 wherein said second surface is an occlusion surface and said second force is an occlusion force applied to an ulnar artery.

3. The closure device of claim 2 wherein said second surface applies said occlusion force adjacent the ulnar artery to totally occlude blood flow through the ulnar artery.

4. The closure device of claim 2 wherein said second surface applies said occlusion force adjacent the ulnar artery to partially occlude blood flow through the ulnar artery.

5. The closure device of claim 2 further comprising a third surface, said third surface being attached to a third support plate, said third surface being attached to a third element that supplies a third force to said third surface to restrict blood flow in the radial artery at a location upstream of the radial artery access site without totally occluding blood flow though the radial artery.

6. The closure device of claim 1 wherein said compression element comprises a first threaded screw that applies said compression force onto said compression surface, and said second element comprises a second threaded screw that applies said second force onto said second surface.

7. The closure device of claim 1 wherein said compression element comprises one or more balloons that apply said compression force onto said compression surface and said second element comprises one or more balloons that apply said second force onto said second surface.

8. The closure device of claim 1 wherein said first support plate and said second support plate are contiguous with each other.

9. The closure device of claim 1 further comprising an energy transducer said energy transducer directing an energy source between said energy transducer and the radial artery downstream of the radial artery access site; said energy transducer indicating the presence of blood flow in the radial artery.

10. The closure device of claim 9 wherein said energy transducer is an ultrasound transducer.

11. The closure device of claim 9 wherein said energy transducer is taken from a group that includes audible pressure signals, electromagnetic energy, laser energy, thermal energy, magnetic signals, and visual signals.

12. The method of use for a radial artery closure device used to control hemostasis at a radial artery access site comprising the steps,
A. placing a support plate across the anterior surface of the forearm at the location of the radial access site,
B. applying an occlusion force to an occlusion element to force an occlusion surface to at least partially occlude the ulnar artery,
C. applying a compression force to a compression element to provide hemostasis at a radial artery access site.

13. The method of claim 12 further comprising the steps,
A. reducing the occlusion force from the occlusion element,
B. reducing the force from the compression element,
C. removing the support plate from the forearm.

14. The method of claim 12 further comprising the steps,
A. activating an energy transducer to direct a signal toward the radial artery downstream from the access site,
B. receiving a signal from said energy transducer indicative of blood flow in the radial artery,
C. adjusting the occlusion element or compression element to maintain radial artery patency and provide hemostasis at the radial access site.

15. The method of claim 12 further comprising the steps,
A. applying a force to a restriction element to force a restriction surface to restrict flow in the radial artery upstream of the access site.

16. The method of claim 15 further comprising the steps,
A. activating an energy transducer to direct a signal toward the radial artery downstream from the access site,
B. receiving a signal from the radial artery indicative of blood flow in the radial artery,
C. adjusting the occlusion means, compression means, or restriction means to maintain radial artery patency and provide hemostasis at the radial access site.

17. A hemostasis device for providing hemostasis at an access site in a first artery that provides blood flow to a distal region of the body, the distal region having a second artery providing blood flow thereto, the first and second arteries having collateral blood vessels connecting the first and second arteries together downstream of the access site, said device comprising;
A. A compression element configured to compress and not totally occlude the first artery and provide hemostasis at the access site,
B. An occlusion element configured to occlude blood flow in the second artery,
C. Said compression element providing an adjustable compression force onto the first artery to provide hemostasis at the access site, and said occlusion element providing an adjustable compression force onto the second artery to improve hemostasis at the access site.
